(19) United States
(12) Patent Application Publication
(10) Pub. No.: US 2005/0245888 A1
(43) Pub. Date: Nov. 3, 2005

(54) Combined peristaltic and vacuum aspiration cassette

(76) Inventor: Laurence J. Cull, Wildwood, MO (US)

Correspondence Address: Michael L. Smith;Bausch & Lomb Incorporated, One Bausch & Lomb Place, Rochester, NY, 14604-2701, US

(21) Appl. No.: 10/834,588

(22) Filed: Apr. 29, 2004

(51) Int. Cl. A61M 1/00

(52) U.S. Cl. 604/317

(57) ABSTRACT

An aspiration collection cassette 10 includes a housing 12 for receiving aspiration fluids from a surgical site. An aspiration port 14 is attached to the housing 12 for connection to an aspiration tube 50 and for providing a passage way to an interior 22 of the housing 12. A vacuum port 16 is disposed in the housing 12 and communicates with the housing interior 22 for cooperation with a vacuum pump. A length of tubing 20 is connected to the housing 12 on each end, such that the tubing 20 will be placed between a peristaltic pump head and a backing plate.

### BACKGROUND OF THE INVENTION

1. Field of the Invention

The present invention is related to a disposable aspiration cassette for use in ophthalmic surgery. More specifically, the present invention is directed to a cassette, which will accommodate both peristaltic and vacuum aspiration of fluids from a surgical site.

2. Description of Related Art

In ophthalmic surgery, two (2) different types of aspiration systems are commonly used during ophthalmic surgery—peristaltic and vacuum systems. The peristaltic pump is well known in the art, and generally includes a plurality of rollers that rotate about a backing plate and pinch-off sections of tubing, as the rollers are rotated, in order to pump fluid from a surgical site into a collection reservoir typically a collapsible bag. Known vacuum systems include venturi systems or rotary-vane pumps, which apply a vacuum to a rigid-sided cassette. The creation of the vacuum within the rigid cassette causes fluid to be pumped from the surgical site through aspiration tubing and into the cassette.

Presently, surgeons need to choose before surgery which type of aspiration system is to be used for a particular surgery to be undertaken. For different types of surgery, a surgeon may prefer a different type of aspiration system depending on his preferences and the type of surgery to be performed. Each type of aspiration system has its advantages and disadvantages, which must be weighed by the surgeon in making his choice of aspiration systems. If a surgeon were to use two (2) types of aspiration systems in one surgery, a collection cassette or bag would need to be used for each pump, as well as separate connections made to the surgical instruments to be used by the surgeon.

In addition, presently known systems typically require a user to thread an irrigation line into a control console, such that a pinch valve or other control may be applied to the irrigation line to control the flow of irrigation fluid from a irrigation source, such as a BSS (balanced-salt solution) bottle, to a handpiece and ultimately to the surgical site. This threading of the irrigation line can be cumbersome and time consuming in an operation room where a reduction in surgery time or surgery set-up time can be quite valuable to a surgeon.

Therefore, it would be desirable to have an aspiration collection cassette, which incorporates the capability of accommodating both a peristaltic pump and a vacuum pump. In addition, it would be advantageous to incorporate an irrigation fluid chamber into the cassette for easy set-up for surgery.

### BRIER DESCRIPTION OF DRAWINGS

FIG. 1 is a perspective drawing of a cassette in accordance with the present invention;

FIG. 2 is a side-elevation view of FIG. 1; and

FIG. 3 is a side-elevation view of a cassette in accordance with the present invention shown in use in vacuum mode.

### DETAILED DESCRIPTION OF THE PREFERRED EMBODIMENT

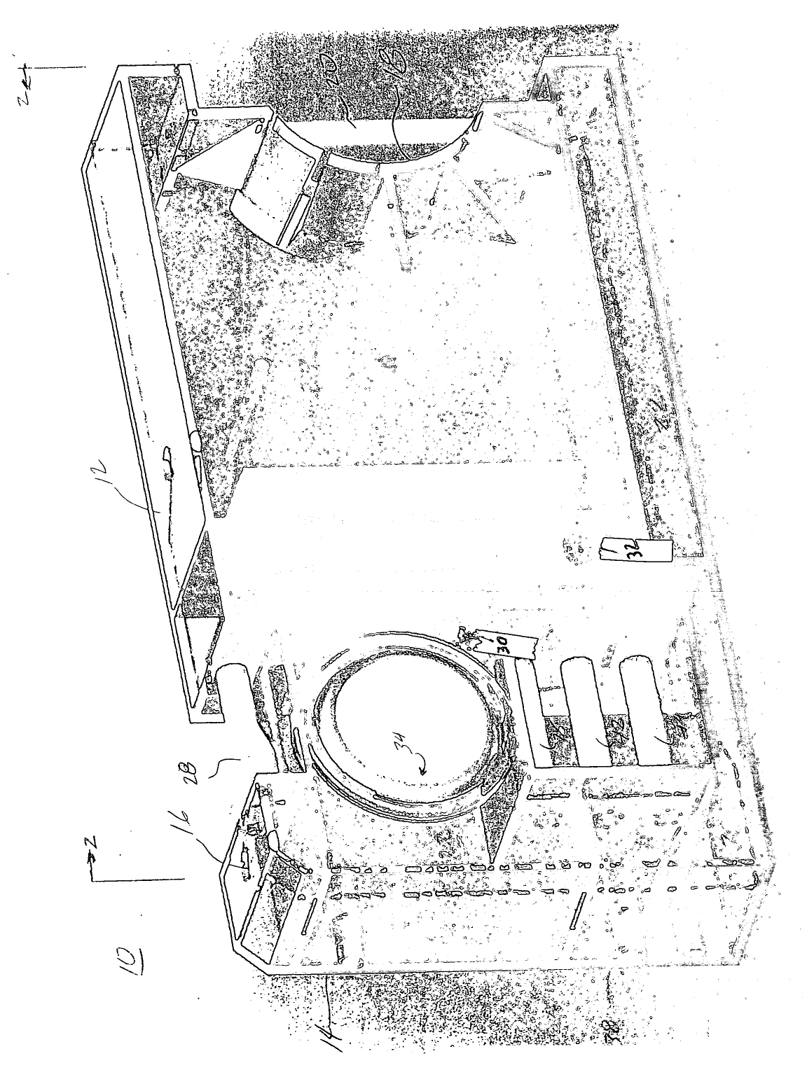

As aspiration collection cassette 10 is shown in FIG. 1. Cassette 10 includes a housing 12 for receiving aspiration fluids from a surgical site (not shown). An aspiration port 14 is attached to the housing 12 for connection to an aspiration tube (not shown) and for providing a passageway to an interior of the housing 12. Preferably, housing 12 is formed of a rigid material, such as polystyrene or other suitable material and is at least partially transparent, as shown in FIG. 1. Obviously, housing 12 does not need to be transparent. A vacuum port 16 is disposed in the housing 12 and is in communication with the housing interior and is for cooperation with a vacuum pump (not shown). A curved portion 18 is preferably formed in the housing 12 and forms at least a portion of a backing plate for rollers of a peristaltic pump (not shown), as is well known in the art. A length of tube 20 is connected to the housing 12 on each end as shown, and positioned such that the tubing 20 will be pinched between the curved portion 18 and the rollers for peristaltically pumping aspirant fluids from a surgical site.

While the backing plate or curved portion 18 is shown as being formed in cassette 10, those skilled in the art will also appreciate that cassette 10 could also include only a loop of tubing 20, with the backing plate being contained within a peristaltic pump console (not shown).

As mentioned above, cassette 10 is preferably formed from a rigid material so that the housing 12 will not collapse when connected to a vacuum pump and used in a vacuum mode.

Cassette 10 also preferably includes, within the housing 12, a vacuum chamber, shown generally at number 22. The vacuum chamber 22 is smaller in volume than a collection chamber, shown generally at 24 within the housing 12. This smaller vacuum chamber 22 provides the advantage of faster response time in vacuum mode, as is taught in commonly owned U.S. Pat. No. 4,626,248, which is hereby incorporated by reference.

The cassette 10 also preferably includes a vacuum chamber conduit 26 formed in the vacuum chamber 22 for cooperation with a pinch valve (not shown). When cassette 10 is used in vacuum mode, vacuum chamber conduit 26 is left in an open condition. However, when cassette 10 is used in flow or peristaltic mode, conduit 26 is preferably pinched shut. This is so that vacuum chamber 22 may become completely filled with fluid so that peristaltic pumping action will function properly. As those skilled in the art will appreciate, when cassette 10 is used in flow mode a vacuum port conduit 28 formed in the housing 12 will cooperate with a pinch valve (not shown) for isolating the vacuum port 16 during operation of the peristaltic pump. This closing of conduit 28 prevents aspiration fluid from flowing into the vacuum pump and contaminating the pump.

Cassette 10 also preferably includes first and second floats 30 and 32. The first float 30 is associated with the vacuum chamber 22 and the second float 32 is associated with the collection chamber 24. These floats 30 and 32 indicate the fluid level of the cassette and may be easily observed by a user and are preferably sensed by an optical sensor (not shown), as is well known in the art, in order to stop the pumps to prevent an overflow of the fluid out of the cassette 10.

Cassette 10 also preferably includes a pressure transducer 34 connected to the housing 12 as shown, for determining a pressure within the vacuum chamber 22. Pressure transducer 34 is preferably a diaphragm transducer, such as shown and is well known in the art.

Cassette 10 also preferably, but not necessarily, includes an irrigation chamber, shown generally at 36 and formed within the housing 12 including an inlet 38 and an outlet 40. Cassette 10 also preferably includes a fluid-venting conduit 42 connected between the irrigation chamber 36 and the vacuum chamber 22, as shown. Fluid venting conduit 42 is preferably a compressible tube as shown, or may be other compliant material which cooperates with a pinch valve (not shown) for venting fluid into vacuum chamber 22, upon the occurrence of an occlusion in the aspiration line, which results in an increased vacuum level in the vacuum chamber 22. Cassette 10 also preferably includes an irrigation conduit 44, which forms a part of the irrigation chamber 36 and cooperates with a pinch valve (not shown) for controlling a flow of irrigation fluid out of the irrigation chamber 36.

Irrigation chamber 36 and pinch valve tubes 42 and 44 provide significant advantages compared to the prior art. The prior art required an irrigation line to be separately threaded into a control console for connection with a pinch valve to open and close the irrigation flow. The prior art is difficult and cumbersome compared to the easy insertion of cassette 10 into a control or pump console, which is automatically aligned with a pinch valve to cooperate with tubing 44. This is also true of fluid venting conduit 42. No threading of the tubing into a control console is needed. In addition, rigid chamber 36 is believed to provide protection against unwanted post occlusion surge by providing a rigid chamber, which will not collapse when a vacuum is applied to the irrigation line from the surgical site.

While conduits 26, 42, and 44 are shown as three separate compliant tubes, those skilled in the art will appreciate that each conduit 26, 42, and 44 may be formed as a single compliant member to allow for ease of assembly and manufacture.

It is noted that vacuum chamber 22 should be minimized in its volume as much as possible, in order to allow for good response from the pumping systems to be used.

It is also noted that, while the inventive irrigation chamber 36 and associated pinch valve tubes 42 and 44 have been shown in connection with a cassette having accommodation for both vacuum and peristaltic pumps, such an irrigation chamber could easily be incorporated into a collection cassette having pump structure for cooperation with a pumping source dedicated to only one type of aspiration system. It is also noted that while conduits 26, 28, 42, and 44 are shown as compliant tubes to be used as pinch valves, other types of valving systems could be incorporated into the cassette. Such other valves could include baffles that would be manipulated by mating mechanisms in a pump console containing cassette 10.

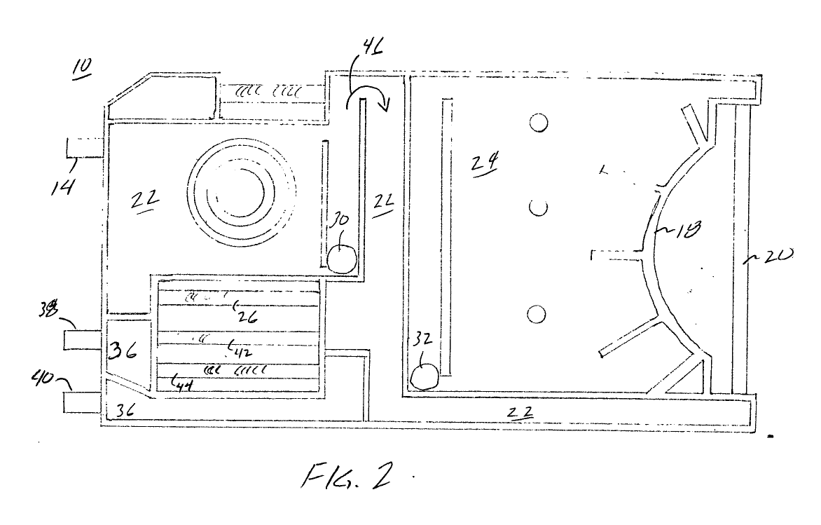

FIG. 2 is an elevation view taken along line 2-2 of FIG. 1. Cassette 10 in the view of FIG. 2 shows aspiration port 14 and inlet 38 and outlet 40. Vacuum chamber 22 and collection chamber 24 are more easily seen compared to FIG. 1. Floats 30 and 32 are also easily seen.

During operation of the cassette, in flow mode, conduit 26 is pinched-off such that aspiration fluid fills vacuum chamber 22 and spills over into another portion of vacuum chamber 22, as shown at arrow 46. In this way vacuum chamber 22 will become completely filled with aspiration fluid so as to eliminate any air bubbles in the aspiration path. This allows for proper functioning of the system in flow mode.

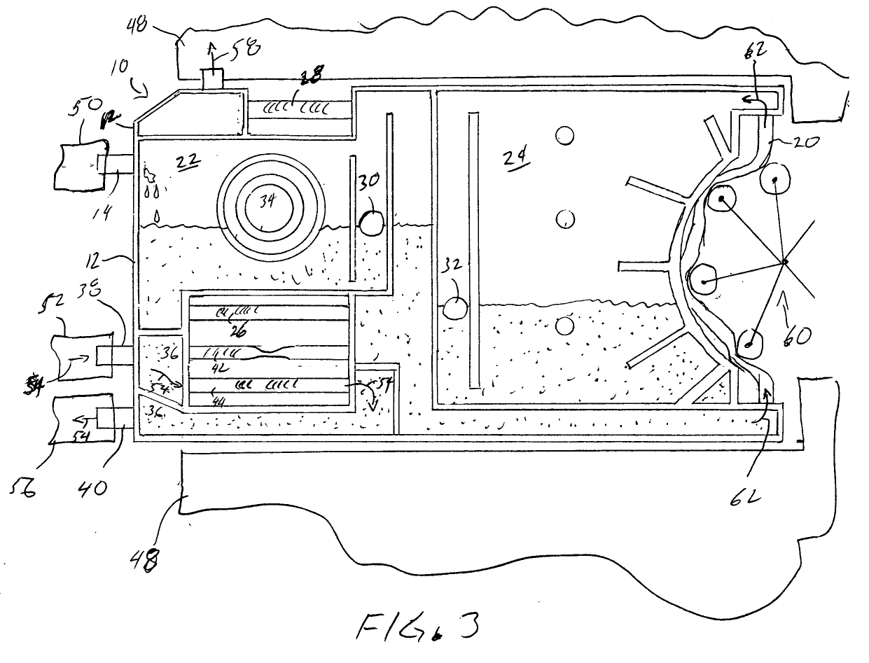

FIG. 3 shows cassette 10 inserted in a control or pump console shown partially at 48. Aspiration port 14 is shown connected to aspiration tube 50. Irrigation-inlet 38 is connected to irrigation line 52, which is connected to an irrigation source (not shown), such as a bottle of balanced salt solution and flows into chamber 36 as indicated at arrow 54. Irrigation fluid is shown, by dots, filling chamber 36 and flows in the direction of arrows 54 through conduit 44 and out outlet 40 and through irrigation line 56 to a surgical handpiece (not shown).

FIG. 3 shows cassette 10, in use in vacuum mode, where a vacuum pump (not shown) draws a vacuum through vacuum port 16 in the direction of arrow 58. Aspiration fluid and excised tissue are drawn through aspiration port 14 into vacuum chamber 22. Conduit 26 is left in an open state so that, as vacuum chamber 22 becomes full, float 30 will cause an optical sensor to activate peristaltic pump 60 to pump fluid through tubing 20 in the direction of arrows 62 into collection chamber 24. When collection chamber 24 reaches capacity, float 32 will cause another optical sensor to shutdown both the vacuum pump and the peristaltic pump 60 to prevent overflow. FIG. 3 also shows fluid venting conduit 42 in a closed or pinched state, which is pinched off by a pinch valve (not shown).

1. An aspiration collection cassette comprising:
a housing for receiving aspiration fluids from a surgical site;
an aspiration port attached to the housing for connection to an aspiration tube and for providing a passage way to an interior of the housing;
a vacuum port disposed in the housing and communicating with the housing interior for cooperation with a vacuum pump;
a curved portion formed in the housing for forming at least a portion of a backing plate for rollers of a peristaltic pump; and
a length of tubing connected to the housing on each end and positioned such that the tubing will be pinched between the curved portion and the rollers for peristaltically pumping aspiration fluids.

2. The cassette of claim 1, wherein the housing is formed of a rigid material so that the housing will not collapse when connected to a vacuum pump.

3. The cassette of claim 1 further including a vacuum chamber within the housing, wherein the vacuum chamber is smaller in volume than a collection chamber within the housing.

4. The cassette of claim 3 further including a vacuum chamber conduit formed in the vacuum chamber and for cooperation with a pinch valve.

5. The cassette of claim 3 further including first and second floats, wherein the first float is associated with the vacuum chamber and the second float is associated with the collection chamber.

6. The cassette of claim 1 further including a vacuum port conduit formed in the housing for cooperation with a pinch valve for isolating the vacuum port during operation of the peristaltic pump.

7. The cassette of claim 1 further including a pressure transducer connected to the housing for determining a pressure within the vacuum chamber.

8. The cassette of claim 1 further including an irrigation chamber formed within the housing including an inlet and an outlet.

9. The cassette of claim 8 further including a fluid venting conduit connected between the irrigation chamber and the vacuum chamber and for cooperation with a pinch valve.

10. The cassette of claim 8 further including an irrigation conduit forming a part of the irrigation chamber for cooperation with a pinch valve for controlling a flow of irrigation fluid out of the irrigation chamber.

11. An aspiration collection cassette comprising:
a housing for receiving aspirant fluids from a surgical site;
an aspiration port attached to the housing for connection to an aspiration tube and for providing a passage way to an interior of the housing;
pump structure formed in the housing for cooperation with a pumping source; and
an irrigation chamber formed within the housing including an inlet and an outlet for receiving irrigation fluid.

12. The cassette of claim 11, wherein the pump structure includes at least a vacuum port disposed in the housing and communicating with the housing interior for cooperation with a vacuum pump.

13. The cassette of claim 11, wherein the pump structure includes at least a length of tubing connected to the housing on each end, such that the tubing will be placed between rollers of a peristaltic pump and a backing plate of the peristaltic pump.

14. The cassette of claim 11 further including a fluid venting conduit connected between the irrigation chamber and another chamber within the housing and for cooperation with a pinch valve.

15. The cassette of claim 11 further including an irrigation conduit forming a part of the irrigation chamber for cooperation with a pinch valve for controlling a flow of irrigation fluid out of the irrigation chamber.

16. The cassette of claim 11 further including at least a portion of a fluid venting valve connected between the irrigation chamber and another chamber within the housing and for cooperation with a control console.

17. The cassette of claim 11 further including at least a portion of an irrigation valve forming a part of the irrigation chamber for cooperation with a control console for controlling a flow of irrigation fluid out of the irrigation chamber.

18. An aspiration collection cassette comprising:
a housing for receiving aspiration fluids from a surgical site;
an aspiration port attached to the housing for connection to an aspiration tube and for providing a passage way to an interior of the housing;
a vacuum port disposed in the housing and communicating with the housing interior for cooperation with a vacuum pump;
a curved portion formed in the housing for forming a backing plate for rollers of a peristaltic pump;
a length of tubing connected to the housing on each end and positioned such that the tubing will be pinched between the curved portion and the rollers for peristaltically pumping aspiration fluids;
a vacuum chamber within the housing, wherein the vacuum chamber is smaller in volume than a collection chamber within the housing;
a vacuum chamber conduit formed in the vacuum chamber and for cooperation with a pinch valve;
first and second floats, wherein the first float is associated with the vacuum chamber and the second flat is associated with the collection chamber;
a vacuum port conduit formed in the housing for cooperation with a pinch valve for isolating the vacuum port during operation of the peristaltic pump;
a pressure transducer connected to the housing for determining a pressure within the vacuum chamber;
an irrigation chamber formed within the housing including an inlet and an outlet;
a fluid-venting conduit connected between the irrigation chamber and the vacuum chamber and for cooperation with a pinch valve; and
an irrigation conduit forming a part of the irrigation chamber for cooperation with a pinch valve for controlling a flow of irrigation fluid out of the irrigation chamber.

19. An aspiration collection cassette comprising:
a housing for receiving aspiration fluids from a surgical site;
an aspiration port attached to the housing for connection to an aspiration tube and for providing a passage way to an interior of the housing;
a vacuum port disposed in the housing and communicating with the housing interior for cooperation with a vacuum pump; and
a length of tubing connected to the housing on each end, such that the tubing will be placed between a peristaltic pump head and a backing plate.

20. The cassette of claim 19, wherein a curved portion is formed in the housing for forming at least a portion of the backing plate.

21. The cassette of claim 19, wherein the housing is formed of a rigid material so that the housing will not collapse when connected to a vacuum pump.

22. The cassette of claim 19 further including a vacuum chamber within the housing, wherein the vacuum chamber is smaller in volume than a collection chamber within the housing.

23. The cassette of claim 22 further including a vacuum chamber conduit formed in the vacuum chamber and for cooperation with a pinch

24. The cassette of claim 22 further including first and second floats, wherein the first float is associated with the vacuum chamber and the second flat is associated with the collection chamber.

25. The cassette of claim 19 further including a vacuum port conduit formed in the housing for cooperation with a pinch valve for isolating the vacuum port during operation of the peristaltic pump.

26. The cassette of claim 19 further including a pressure transducer connected to the housing for determining a pressure within the vacuum chamber.

27. The cassette of claim 19 further including an irrigation chamber formed within the housing including an inlet and an outlet.

28. The cassette of claim 27 further including a fluid venting conduit connected between the irrigation chamber and the vacuum chamber and for cooperation with a pinch valve.

29. The cassette of claim 27 further including an irrigation conduit forming a part of the irrigation chamber for cooperation with a pinch valve for controlling a flow of irrigation fluid out of the irrigation chamber.

30. The cassette of claim 19 further including at least a portion of a fluid venting valve connected between the irrigation chamber and another chamber within the housing and for cooperation with a control console.

31. The cassette of claim 19 further including at least a portion of an irrigation valve forming a part of the irrigation chamber for cooperation with a control console for controlling a flow of irrigation fluid out of the irrigation chamber.
